(19) United States
(12) Patent Application Publication
(10) Pub. No.: US 2021/0386752 A1
(43) Pub. Date: Dec. 16, 2021

(54) Uses of Electron Transport Chain Complex I or Complex II Inhibitors in Treating Cancer, Combination Therapies, and Diagnostic Methods Related Thereto

(71) Applicant: Emory University, Atlanta, GA (US)

(72) Inventors: Malathy Shanmugam, Atlanta, GA (US); Aditi Sharma, Atlanta, GA (US)

(21) Appl. No.: 17/348,487

(22) Filed: Jun. 15, 2021

Related U.S. Application Data
(60) Provisional application No. 63/039,227, filed on Jun. 15, 2020.

(51) Int. Cl. A61K 31/5395; A61K 31/635; A61K 31/573; A61K 31/198; A61K 31/4965; A61K 31/5377

(52) U.S. Cl. CPC A61K 31/5395; A61K 31/635; A61K 31/5377; A61K 31/198; A61K 31/4965; A61K 31/573

(57) ABSTRACT

In certain embodiments, this disclosure relates to methods of treating cancer comprising administering an electron transport chain complex I inhibitor and/or a complex II succinate ubiquinone reductase (SQR) inhibitor in combination with another anticancer agent such as a BCL-2 antagonist or proteasome inhibitor. In certain embodiments, the cancer is a hematological cancer such as multiple myeloma, chronic lymphocytic leukemia, or acute myeloid leukemia. In certain embodiments, the treatment is under low glucose or fasting conditions.

### CROSS-REFERENCE TO RELATED APPLICATIONS

This application claims the benefit of U.S. Provisional Application No. 63/039,227 filed Jun. 15, 2020. The entirety of this application is hereby incorporated by reference for all purposes.

### STATEMENT REGARDING FEDERALLY SPONSORED RESEARCH OR DEVELOPMENT

This invention was made with government support under CA208328 awarded by the National Institutes of Health. The government has certain rights in the invention.

### BACKGROUND

Multiple myeloma (MM) is a plasma cell malignancy. Despite present chemotherapy strategies which can extend survival, many patients succumb to this disease. Relapse and the development of refractory disease occur primarily due to the emergence of chemotherapy resistant cells. Thus, there is a need to identify improved treatments.

Cancerous cells evade a self-destruction mechanism referred to as apoptosis. The BCL-2 family of proteins regulate apoptosis. The BCL-2 family proteins are grouped into three sub-families based on the number of BCL-2 Homology (BH) domains. The first includes four BH domains, e.g., BCL-2, BCL-xL, BCL-w, MCL-1, and A1/BFL-1). The next group possesses three BH domains, e.g., BAX, BAK, and BOK. The last group termed “BH3-only proteins” is characterized by the presence of only a BH3 domain. See Lomonosov et al. BH3-only proteins in apoptosis and beyond: an overview, Oncogene, 2008, 27 (Suppl 1): S2-19. Certain BH3-only proteins have the ability to activate effector proteins BAX and/or BAK which oligomerize and permeabilize the outer mitochondrial membrane releasing cytochrome c to activate subsequent steps of apoptosis resulting in cell death.

The BH3-only protein, BIM, is expressed in cells of hematopoietic origin and binds to multiple proteins such as BCL-2, MCL-1, BCLxL and BAX. BH3 mimetics, which block the interaction of BIM-BCL-2, sometimes promote apoptosis. For example, venetoclax is a BCL-2 antagonist effective in BCL-2-dependent malignancies and approved for the treatment of chronic lymphocytic leukemia (CLL) and acute myeloid leukemia (AML).

In multiple myeloma, the malignant plasma cells commonly contain a t(11;14)(q13;q32) chromosome translocation. Only a small fraction of multiple myeloma patients exhibiting the 11;14 translocation, respond to venetoclax as a single agent. Thus, there is need for identifying improved treatment strategies for multiple myeloma.

Bajpai et al. report targeting glutamine metabolism in multiple myeloma enhances BIM binding to BCL-2 eliciting synthetic lethality to venetoclax. Oncogene, 2016, 35, 3955-3964.

Bajpai et al. report electron transport chain activity is a predictor and target for venetoclax sensitivity in multiple myeloma. Nature Communications, volume 11, Article number: 1228 (2020)

Shanmugam et al. report methods of treating cancer with a combination of glucose modulators and BCL-2 inhibitors. US 2016/0113925.

Guieze et al. report regulators of lymphoid transcription and cellular energy metabolism increase in MCL-1, activation of AMPK and OXPHOS as drivers underlying resistance to BCL-2 inhibition in lymphoid malignancies. Cancer Cell, 2019, 36, 369-384.

References cited herein are not an admission of prior art.

### SUMMARY

In certain embodiments, this disclosure relates to methods of treating cancer comprising administering an electron transport chain complex I inhibitor and/or a complex II succinate ubiquinone reductase (SQR) inhibitor or succinate dehydrogenase (SDHA) inhibitors that reduce SQR activity in combination with another anticancer agent such as a BCL-2 antagonist or proteasome inhibitor. In certain embodiments, the cancer is a hematological cancer such as multiple myeloma, chronic lymphocytic leukemia, or acute myeloid leukemia. In certain embodiments, the treatment is under low glucose or fasting conditions.

In certain embodiments, the electron transport chain complex I inhibitor is 5-(5-methyl-1-(3-(4-(methylsulfonyl)piperidin-1-yl)benzyl)-1H-1,2,4-triazol-3-yl)-3-(4-(trifluoromethoxy) phenyl)-1,2,4-oxadiazole (IACS-010759), its derivative, prodrug, or salts thereof.

In certain embodiments, the BCL-2 antagonist is 2-((1H-pyrrolo[2,3-b]pyridin-5-yl)oxy)-4-(4((4′-chloro-5,5-dimethyl-3,4,5,6-tetrahydro-[1,1′-biphenyl]-2-yl)methyl)piperazin-l-yl)-N-((3-nitro-4-(((tetrahydro-2H-pyran-4-yl)methyl)amino)phenyl)sulfonyl)benzamide (venetoclax), its derivative, prodrug, or salts thereof.

### BRIEF DESCRIPTION OF THE SEVERAL VIEWS OF THE DRAWINGS

FIGS. 1A-1E show data indicating venetoclax-sensitive MINI exhibits reduced cellular energetics in contrast to the venetoclax-resistant cells.

FIG. 1A shows data where MM cell lines were treated ±0.5 μM venetoclax (Ven) for 24 h were assessed for cell death by AnnexinV/4′,6-diamidino-2-phenylindole (DAPI) flow cytometric staining. Percent live normalized to vehicle control, with cell lines grouped by sensitivity. n=3 independent experiments.

FIG. 1B shows data where MINI lines were evaluated for basal, coupled and maximal respiration in a stress assay using a Seahorse XFe96 analyzer. n=6 replicate Seahorse wells for each cell line except JJN3 (n=11), U266 (n=8) and KMS21BM (n=7). OCR displayed are normalized to live cell number. Venetoclax-sensitive MM exhibited significantly lower basal, maximal and coupled OCR determined after the addition of oligomycin, FCCP and antimycin/rotenone.

FIG. 1C shows data on coupled respiration.

FIG. 1D shows data on maximal respiration.

FIG. 1E shows data on spare respiratory capacity. Spare capacity in venetoclax-sensitive and venetoclax -resistant cells was determined by subtracting basal OCR from maximal OCR.

FIGS. 2A-2D show data indicating venetoclax-sensitive MM exhibits reduced Complex I and Complex II activity. Complex I, SQR and SDH activity were significantly different in sensitive vs. resistant lines while CS activity did not differ significantly.

FIG. 2A shows data where Complex I activity was assessed.

FIG. 2B shows data on SDH activity assessed in gently permeabilized whole cells supplemented with succinate and Complex I, III and IV inhibitors.

FIG. 2C shows data on SQR activity.

FIG. 2D shows data on CS activity.

FIGS. 3A-3K shows data on the inhibition of SQR with Qp site inhibitor TTFA effectively sensitizes resistant MM to venetoclax.

FIG. 3A shows data on SQR inhibition with TTFA (100 μM) indicating it sensitizes myeloma cell line L363 to venetoclax more effectively than inhibition of SDHA with 3-NPA upon cotreatment with indicated doses of venetoclax for 24 h. Cell viability assessed by AnnexinV/DAPI staining.

FIG. 3B shows data using myeloma cell line JJN3.

FIG. 3C shows data using myeloma cell line MM.1S.

FIG. 3D shows data using myeloma cell line RPMI-8226.

FIG. 3E shows data using myeloma cell line KMS18.

FIG. 3F shows data using myeloma cell line KMS11.

FIG. 3G shows data using myeloma cell line U266.

FIG. 3H shows data using myeloma cell line KMS21BM.

FIG. 3I shows data on SQR activities demonstrating selective inhibition of SQR activity upon 24 h TTFA treatment.

FIG. 3J shows data on SDH.

FIG. 3K shows data on CS.

FIGS. 4A-4E show data indicating SQR inhibition via Qp site SDHC-mutant introduction sensitizes MM to venetoclax.

FIG. 4A shows data on SQR activity determined in KMS11 and L363, SDHCKO cells expressing SDHC-WT or SDHC-R72C mutant constructs.

FIG. 4B shows data on SDH activity.

FIG. 4C shows data on CS activity.

FIG. 4D shows data on SDHC-WT and SDHC-R72C mutant-expressing cells ±0.5 μM venetoclax (24 h) which were evaluated for viability by Annexin V/DAPI flow cytometric staining, demonstrating increased sensitivity of SDHC-R72C cells to venetoclax.

FIG. 4E shows data on spare respiratory capacity determined in the indicated SDHC-WT and SDHC-R72C mutant-expressing cells indicating a net reduction in SRC upon introduction of the mutant.

FIG. 5A-5O show data indicating ATF4, BIM and NOXA regulate TTFA-induced venetoclax sensitivity in MM.

FIG. 5A shows data on the expression of ATF4 and the indicated pro- and antiapoptotic proteins evaluated in whole cell lysates of the indicated lines treated with ±100 μM TTFA for 24 h with Actin as loading control. Representative blots from one of three independent experiments is presented.

FIG. 5B shows data on protein expression levels evaluated in SDHC-WT or SDHC-R72C mutant-expressing cells.

FIG. 5C shows data from control siRNA or ATF4 siRNA transfected JJN3 cells treated with venetoclax (0.5 μM), TTFA (100 μM) or the combination for 24 h and cell viability assessed by Annexin V/DAPI flow cytometric staining.

FIG. 5D shows data for KMS11 cells.

FIG. 5E shows data for cells in FIG. 5C and 5D used to prepare lysates for immunoblot evaluation of indicated proteins.

FIG. 5F shows data from CRISPR Cas9 generated KMS11 knockout treated with ±TTFA (100 μM), ±venetoclax (0.5 μM) for 24 h and cell death evaluated by Annexin V/DAPI flow cytometric staining.

FIG. 5G shows data for L363 cells.

FIG. 5H shows data on BIMKO (KO efficiency) for KMS11 cells.

FIG. 5I shows data on BIMKO (KO efficiency) for L363 cells.

FIG. 5J shows data on whole-cell lysates from KMS11 and KMS21BM treated or untreated with TTFA evaluated for expression of indicated proteins by immunoblot analysis.

FIG. 5K shows data on cellular lysates evaluated for immunoprecipitates of BCL-2 and bound BIM by immunoblotting.

FIG. 5L shows data on whole-cell lysates from KMS18 NOXA KO cells treated or untreated with TTFA were evaluated for expression of indicated proteins by immunoblot analysis.

FIG. 5M shows data for KMS18 NOXA KO cells treated with ±TTFA (100 μM), ±venetoclax (0.5 μM) for 24 h and cell death evaluated by Annexin V/DAPI flow cytometric staining.

FIG. 5N shows data on whole-cell lysates from RPMI-8226 cells treated or untreated with TTFA were evaluated for expression of indicated proteins by immunoblot analysis.

FIG. 5O shows data for RPMI-8226 cells treated with ±TTFA (100 μM), ±venetoclax (0.5 μM) for 24 h and cell death evaluated by Annexin V/DAPI flow cytometric staining.

FIGS. 6A-6C show data indicating SQR activity inversely correlates with venetoclax sensitivity in MM patient samples.

FIG. 6A shows a box plot of cell death measured by Annexin V staining relative to vehicle control in samples from 50 myeloma patient bone marrow aspirates treated with 0.1 μM venetoclax, 100 μM TTFA alone or in the combination for 24 h. CD38-PE and CD45-APC-Cy7 were used to gate myeloma cells. n=50 biological independent samples. Boxplots show the median and quartiles with the whiskers extending to the most extreme data point within 1.5 times the interquartile range.

FIG. 6B shows a scatter plot of IC₅₀ values for patient samples treated with venetoclax (Ven; x-axis) vs. those treated with Ven and 100 μM TTFA (y-axis). A diagonal line denotes one-to-one correspondence of IC₅₀. The dashed box denotes patient samples with Ven IC₅₀ >100 nM and Ven+ TTFA IC_{50S}≤100 nM. Samples with an IC₅₀<1 nM are plotted at 1 nM.

FIG. 6C shows a scatter plot of SQR activity and Venetoclax IC₅₀ showing a positive correlation (Spearman's rank correlation (ρ)=0.824, n=14). Triangles denote t(11;14) samples and circles denote non-t(11;14) samples. The dashed box denotes patient samples with Ven IC₅₀≤0.1 μM and SQR activity ≤0.25 nmol min⁻¹ mL⁻¹.

FIG. 7A shows data indicating inhibition of Complex I with IACS-010759 sensitizes resistant MM to venetoclax. Box plot of ICso values of Ven and Ven+IACS, and table with FISH characteristics of nine myeloma patient samples. Boxplots show the median and quartiles with the whiskers extending to the most extreme data point within 1.5 times the interquartile range. PS10001243 was resistant to Ven±IACS and has been represented to have an artificial IC₅₀ of 100 μM for Ven±IACS in the box plot.

FIG. 7B illustrates a mechanistic representation of how Complex I and Complex II regulate BCL-2 dependence in an MINI cell. IACS-010759 and TTFA inhibit Complex I and Complex II, respectively, resulting in ETC inhibition. ETC inhibition regulates BCL-2 dependency by inducing ATF4. ATF4 induces NOXA that displaces BIM from MCL-1. The increased binding of BIM to BCL-2 elevates BCL-2 dependence leading to sensitization to venetoclax. It is not intended that certain embodiments of this disclosure be limited by any particular mechanism.

FIG. 8A shows data indicating IACS-010759 increases efficacy of dexamethasone (Dex) +Ven in MM (Venetoclax given in combination with Dex to t(11;14) myeloma patients).

FIG. 8B shows data indicating IACS-010759 increases sensitivity of MM to Dex.

FIG. 8C shows data indicating IACS-010759 sensitizes t(11;14) MM to venetoclax (ABT-199) and IACS-010759 reduces MCL-1 (pro-survival BCL-2 family member) expression in MM, KMS12PE, 24 hr Ven treatment.

FIG. 9 shows data indicating IACS-010759 sensitizes myeloma to melphalan induced cell death, L363, 24 hr treatment.

FIG. 10 shows data indicating IACS-010759 antagonizes MM to proteasome inhibitor bortezomib induced cell death, L363, 24 hr treatment.

FIG. 11 shows data indicating IACS-010759 antagonizes MM to proteasome inhibitor carfilzomib (CFZ), 24 hr dose response.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Certain of the compounds described herein may contain one or more asymmetric centers and may give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, tautomer forms, hydrated forms, optically substantially pure forms and intermediate mixtures.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In typical embodiments, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Subject” refers to any animal, preferably a human patient, livestock, rodent, monkey or domestic pet.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis.

Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

As used herein, “pharmaceutically acceptable esters” include, but are not limited to, alkyl, alkenyl, alkynyl, aryl, arylalkyl, and cycloalkyl esters of acidic groups, including, but not limited to, carboxylic acids, phosphoric acids, phosphinic acids, sulfonic acids, sulfinic acids, and boronic acids.

As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom or replacing an amino group with a hydroxyl group or vice versa. Replacing a carbon with nitrogen in an aromatic ring is a contemplated derivative. The derivative may be a prodrug. Derivatives may be prepared by any variety of synthetic methods or appropriate adaptations presented in the chemical literature or as in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“—O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═0)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The cancer to be treated in the context of the present disclosure may be any type of cancer or tumor. These tumors or cancer include, and are not limited to, tumors of the hematopoietic and lymphoid tissues or hematopoietic and lymphoid malignancies, tumors that affect the blood, bone marrow, lymph, and lymphatic system. Hematological malignancies may derive from either of the two major blood cell lineages: myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

A “chemotherapy agent,” “chemotherapeutic,” “anti-cancer agent” or the like, refer to molecules that are recognized to aid in the treatment of a cancer. Contemplated examples include the following molecules or derivatives such as temozolomide, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, idoxifene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin and/or lenalidomide or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

### Methods of Use

In certain embodiments, this disclosure relates to methods of treating cancer comprising administering an electron transport chain complex I inhibitor and/or a complex II succinate ubiquinone reductase (SQR) inhibitor in combination with another anticancer agent such as a BCL-2 antagonist or proteasome inhibitor. In certain embodiments, the cancer is a hematological cancer such as multiple myeloma, chronic lymphocytic leukemia, or acute myeloid leukemia. In certain embodiments, the treatment is under low glucose or fasting conditions.

In certain embodiments, the electron transport chain complex I inhibitor is 5-(5-methyl-1-(3-(4-(methylsulfonyl)piperidin-1-yl)benzyl)-1H-1,2,4-triazol-3-yl)-3-(4-(trifluoromethoxy) phenyl)-1,2,4-oxadiazole (IACS-010759), its derivative, prodrug, or salts thereof.

In certain embodiments, the BCL-2 antagonist is 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)benzamide(venetoclax), its derivative, prodrug, or salts thereof.

In certain embodiments, the BCL-2 antagonist is 4-(4-(4′-chloro-[1,1′-biphenyl]-2-yl)methyl)piperazin-1-yl)-N-((4-((4-(dimethylamino)-1- (phenylthio)butan-2-yl)amino)-3-nitrophenyl)sulfonyl)benzamide (ABT-737), its derivative, prodrug, or salts thereof.

In certain embodiments, the BCL-2 antagonist is 4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-(trifluoromethyl)sulfonyl)phenyl)sulfonyl)benzamide (navitoclax), its derivative, prodrug, or salts thereof.

In certain embodiments, the BCL-2 antagonist is 2-(2-((3,5-dimethyl-1H-pyrrol-2-yl)methylene)-3-methoxy-2H-pyrrol-5-yl)-1H-indole (obatoclax), its derivative, prodrug, or salts thereof.

In certain embodiments, the complex II inhibitor is 4,4,4-trifluoro-1-(2-thienyl)-1,3-butanedione (TTFA), its derivative, prodrug, or salts thereof.

In certain embodiments, the complex II inhibitor is 1-(2,4-dichlorobenzyl)-1H-indazole-3-carboxylic acid (lonidamine), its derivative such as mito-lonidamine, prodrug, or salts thereof.

In certain embodiments, the complex II inhibitor is 2-((3-hydroxy-2-(hydroxymethyl)-6-((5′,6a,8a,9-tetramethyl-1,3,3′,4,4′,5,5′,6,6a,6b,6′,7,8,8a,8b,9,11a,12,12a,12b-icosahydrospiro[naphtho[2′,1′:4,5]indeno[2,1-b]furan-10,2′-pyran]-4-yl)oxy)-5-((3,4,5-trihydroxy-6- methyltetrahydro-2H-pyran-2-yl)oxy)tetrahydro-2H-pyran-4-yl)oxy)-6-(hydroxymethyl)tetrahydro-2H-pyran-3,4,5-triol (gracillin), its derivative, prodrug, or salts thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methylsulfonyl)piperidin-1-yl)benzyl)-1H-1,2,4-triazol-3-yl)-3-(4- (trifluoromethoxy)phenyl)-1,2,4-oxadiazole (IACS-010759) or salt thereof in combination with venetoclax to a subject in need thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methylsulfonyl)piperidin-1-yl)benzyl)-1H-1,2,4-triazol-3-yl)-3-(4- (trifluoromethoxy)phenyl)-1,2,4-oxadiazole (IACS-010759) in combination with venetoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methylsulfonyl)piperidin-1-yl)benzyl)-1H-1,2,4-triazol-3-yl)-3-(4- (trifluoromethoxy)phenyl)-1,2,4-oxadiazole (IACS-010759) or salt thereof in combination with dexamethasone or melphalan.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methylulfonyl)piperidin-1-yl)benzyl)-1H-1,2,4-triazol-3-yl)-3-(4- (trifluoromethoxy)phenyl)-1,2,4-oxadiazole (IACS-010759) or salt thereof in combination with melphalan to a subject in need thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methylsulfonyl)piperidin-1-yl)benzyl)-1H-1,2,4-triazol-3-yl)-3-(4- (trifluoromethoxy)phenyl)-1,2,4-oxadiazole (IACS-010759) in combination with venetoclax and melphalan or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methylsulfonyl)piperidin-1-yl)benzyl)-1H-1,2,4-triazol-3-yl)-3-(4- (trifluoromethoxy)phenyl)-1,2,4-oxadiazole (IACS-010759) or salt thereof in combination with bortezomib to a subject in need thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methylsulfonyl)piperidin-1-yl)benzyl)-1H-1,2,4-triazol-3-yl)-3-(4- (trifluoromethoxy)phenyl)-1,2,4-oxadiazole (IACS-010759) or salt thereof in combination with carfilzomib to a subject in need thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3 -(4-(methylsulfonyl)piperidin-1-yl)benzyl)-1H-1,2,4-triazol-3-yl)-3-(4- (trifluoromethoxy)phenyl)-1,2,4-oxadiazole (IACS-010759) or salt thereof in combination with navitoclax to a subject in need thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3 -(4-(methylsulfonyl)piperidin-1-yl)benzyl)-1H-1,2,4-triazol-3-yl)-3-(4- (trifluoromethoxy)phenyl)-1,2,4-oxadiazole (IACS-010759) in combination with navitoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methylsulfonyl)piperidin-1-yl)benzyl)-1H-1,2,4-triazol-3-yl)-3-(4- (trifluoromethoxy)phenyl)-1,2,4-oxadiazole (IACS-010759) or salt thereof in combination with ABT-737 to a subject in need thereof

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methylsulfonyl)piperidin-1-yl)benzyl)-1H-1,2,4-triazol-3-yl)-3-(4- (trifluoromethoxy)phenyl)-1,2,4-oxadiazole (IACS-010759) in combination with ABT-737 and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methyl sulfonyl)piperidin-1-yl)benzyl)-1H-1,2,4-triazol-3-yl)-3-(4- (trifluoromethoxy)phenyl)-1,2,4-oxadiazole (IACS-010759) or salt thereof in combination with obatoclax to a subject in need thereof.

In certain embodiments, this disclosure relates to methods of treating a hematological cancer such as multiple myeloma, chronic lymphocytic leukemia, or acute myeloid leukemia comprising administering an effective amount of 5-(5-methyl-1-(3-(4-(methyl sulfonyl)piperidin-1-yl)benzyl)-1H-1,2,4-triazol-3-yl)-3-(4- (trifluoromethoxy)phenyl)-1,2,4-oxadiazole (IACS-010759) in combination with obatoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine or salt thereof in combination with venetoclax to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine in combination with venetoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine or salt thereof in combination with dexamethasone or melphalan.

In certain embodiments, this disclosure relates to methods of treating a hematological cancer such as multiple myeloma, chronic lymphocytic leukemia, or acute myeloid leukemia comprising administering an effective amount of lonidamine or salt thereof in combination with melphalan to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine in combination with venetoclax and melphalan or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine or salt thereof in combination with bortezomib to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine or salt thereof in combination with carfilzomib to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine or salt thereof in combination with navitoclax to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine in combination with navitoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine or salt thereof in combination with ABT-737 to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine in combination with ABT-737 and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine or salt thereof in combination with obatoclax to a subject in need thereof.

In certain embodiments, this disclosure relates to methods of treating a hematological cancer such as multiple myeloma, chronic lymphocytic leukemia, or acute myeloid leukemia comprising administering an effective amount of lonidamine in combination with obatoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine or salt thereof in combination with venetoclax to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine in combination with venetoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine or salt thereof in combination with dexamethasone or melphalan.

In certain embodiments, this disclosure relates to methods of treating a hematological cancer such as multiple myeloma, chronic lymphocytic leukemia, or acute myeloid leukemia comprising administering an effective amount of mito-lonidamine or salt thereof in combination with melphalan to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine in combination with venetoclax and melphalan or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine or salt thereof in combination with bortezomib to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine or salt thereof in combination with carfilzomib to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine or salt thereof in combination with navitoclax to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine in combination with navitoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine or salt thereof in combination with ABT-737 to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine in combination with ABT-737 and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine or salt thereof in combination with obatoclax to a subject in need thereof.

In certain embodiments, this disclosure relates to methods of treating a hematological cancer such as multiple myeloma, chronic lymphocytic leukemia, or acute myeloid leukemia comprising administering an effective amount of mito-lonidamine in combination with obatoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of gracillin or salt thereof in combination with venetoclax to a subject in need thereof.

In certain embodiments, this disclosure relates to methods of treating a hematological cancer such as multiple myeloma, chronic lymphocytic leukemia, or acute myeloid leukemia comprising administering an effective amount of gracillin in combination with venetoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of gracillin or salt thereof in combination with dexamethasone or melphalan.

In certain embodiments, this disclosure relates to methods of treating a hematological cancer such as multiple myeloma, chronic lymphocytic leukemia, or acute myeloid leukemia comprising administering an effective amount of gracillin or salt thereof in combination with melphalan to a subject in need thereof.

In certain embodiments, this disclosure relates to methods of treating a hematological cancer such as multiple myeloma, chronic lymphocytic leukemia, or acute myeloid leukemia comprising administering an effective amount of gracillin in combination with venetoclax and melphalan or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of gracillin or salt thereof in combination with bortezomib to a subject in need thereof.

In certain embodiments, this disclosure relates to methods of treating a hematological cancer such as multiple myeloma, chronic lymphocytic leukemia, or acute myeloid leukemia comprising administering an effective amount of gracillin or salt thereof in combination with carfilzomib to a subject in need thereof.

In certain embodiments, this disclosure relates to methods of treating a hematological cancer such as multiple myeloma, chronic lymphocytic leukemia, or acute myeloid leukemia comprising administering an effective amount of gracillin or salt thereof in combination with navitoclax to a subject in need thereof.

In certain embodiments, this disclosure relates to methods of treating a hematological cancer such as multiple myeloma, chronic lymphocytic leukemia, or acute myeloid leukemia comprising administering an effective amount of gracillin in combination with navitoclax and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of gracillin or salt thereof in combination with ABT-737 to a subject in need thereof.

In certain embodiments, this disclosure relates to methods of treating a hematological cancer such as multiple myeloma, chronic lymphocytic leukemia, or acute myeloid leukemia comprising administering an effective amount of gracillin in combination with ABT-737 and dexamethasone or salt thereof to a subject in need thereof.

In certain embodiments, this disclosure relates to methods of treating a hematological cancer such as multiple myeloma, chronic lymphocytic leukemia, or acute myeloid leukemia comprising administering an effective amount of gracillin or salt thereof in combination with obatoclax to a subject in need thereof.

In certain embodiments, this disclosure relates to methods of treating a hematological cancer such as multiple myeloma, chronic lymphocytic leukemia, or acute myeloid leukemia comprising administering an effective amount of gracillin in combination with obatoclax and dexamethasone or salt thereof to a subject in need thereof.

In certain embodiments, this disclosure relates to methods of diagnosing and treating a patient with cancer comprising: measuring succinate ubiquinone reductase (SQR) activity of Complex II, determining a low SQR activity when compared to a reference amount or normal cell, and administering an effective amount of a BCL-2 antagonist to a subject in need thereof. In certain embodiments, the cancer is a hematological cancer such as multiple myeloma or acute myeloid leukemia.

In certain embodiments, the cancer is a hematological cancer such as multiple myeloma or acute myeloid leukemia and administration is a single agent venetoclax monotherapy.

In certain embodiments, this disclosure relates to methods of screening a compound or a library of compounds for an anticancer agent comprising mixing a test compound with succinate and succinate ubiquinone reductase (SQR), analyzing whether the test compound reduces the reduction of succinate, and identifying the test compound that reduces the reduction of succinate as an anticancer agent that promotes apoptosis.

In certain embodiments, the method further comprises administering an effective amount of the anticancer agent that that reduces the reduction of succinate to a subject diagnosed with cancer optionally in combination with another anticancer agent such a BCL-2 antagonist.

In certain embodiments, the patient is instructed not to eat foods that contain sugars or a substantial amount of carbohydrates.

In certain embodiments, the patient is instructed not to eat foods that contain sugars or a substantial amount of carbohydrates for more than one day before administration. In certain embodiments, the patient is instructed not to eat foods that contain sugars or a substantial amount of carbohydrates for more than one day after administration. In certain embodiments, the patient is instructed not to eat foods that contain sugars or a substantial amount of carbohydrates for more than two days before administration. In certain embodiments, the patient is instructed not to eat foods that contain sugars or a substantial amount of carbohydrates for more than two days after administration. In certain embodiments, the patient is instructed not to eat foods that contain sugars or a substantial amount of carbohydrates for more than three days before administration. In certain embodiments, the patient is instructed not to eat foods that contain sugars or a substantial amount of carbohydrates for more than three days after administration.

In certain embodiments, the patient is instructed not to eat foods that contain sugars or a substantial amount of carbohydrates for more than a week before administration. In certain embodiments, the patient is instructed not to eat foods that contain sugars or a substantial amount of carbohydrates for more than a week after administration. In certain embodiments, the patient is instructed not to eat foods that contain sugars or a substantial amount of carbohydrates for more than two weeks before administration. In certain embodiments, the patient is instructed not to eat foods that contain sugars or a substantial amount of carbohydrates for more than two weeks after administration.

In certain embodiments, the patient is instructed not to eat for more than one day before administration. In certain embodiments, the patient is instructed not to eat for more than one day after administration. In certain embodiments, the patient is instructed not to eat for more than two days before administration. In certain embodiments, the patient is instructed not to eat more than two days after administration. In certain embodiments, the patient is instructed not to eat for more than three days before administration. In certain embodiments, the patient is instructed not to eat for more than three days after administration.

### Electron Transport Chain Activity is a Predictor and Target for Venetoclax Sensitivity in Multiple Myeloma

The BH3 activator, BIM, is highly expressed in cells of hematopoietic origin and evaluation of BH3 activators bound to antiapoptotics in MM demonstrated that BIM is the most relevant BH3 activator bound to BCL-2. Despite heavy reliance of MM on MCL-1 and correlation of MCL-1 levels with poor prognoses, targeting BIM-BCL-2 interactions has been shown to induce apoptosis even in MCL-1-dependent MM. BH3 mimetics are a class of small molecules that block the interaction of specific proapoptotics with cognate antiapoptotics, releasing bound proapoptotic activators. Venetoclax is one such selective, potent BCL-2 antagonist. It is highly effective in BCL-2-dependent malignancies and FDA-approved for the treatment of chronic lymphocytic leukemia (CLL) and with hypomethylating agents azacitidine or decitabine (NCT02203773) or low dose cytarabine (NCT02287233) in acute myeloid leukemia (AML). A small fraction (approximately 7%) of MM patients respond to single agent venetoclax. Given the plethora of new myeloma therapies, there is need for precision therapy informed by biomarkers or molecular traits. Understanding the basis for single-agent efficacy of venetoclax in t(11;14) myeloma can be highly informative for identifying patients who will benefit from single-agent venetoclax therapy as well as identify targets for developing rational venetoclax containing combinations to expand use of venetoclax beyond the small cohort of patients currently sensitive to venetoclax monotherapy. Metabolites regulate the primed state, i.e. proximity to the apoptotic threshold, by regulating the expression and binding properties of pro- and antiapoptotic BCL-2 family members. Metabolites such as glucose, glutamine and (R)-2HG22 regulate BCL-2, MCL-1, BCL-xL, PUMA, NOXA and BIM expression and/or their interactions. Perturbing metabolism can alter dependence and sensitivity to specific BH3 mimetics. Glutamine deprivation increases BIM binding to BCL-2 thereby sensitizing MM to venetoclax while supplementation with α-ketoglutarate reversed this sensitivity, affirming metabolic regulation of BCL-2 dependence. Experiments where performed to determine how the presence of a metabolic basis for t(11;14) myeloma sensitivity to single-agent venetoclax that could aid in identifying (1) venetoclax-sensitive MM in the broader MM population, and (2) metabolic targets that could be inhibited to sensitize resistant MM to venetoclax. These experiments indicate that Complex I and the succinate ubiquinone reductase (SQR) activity of Complex II of the ETC as targets for venetoclax sensitization and SQR activity as an assessable predictor of patient response. MM cells expressing the t(11;14) translocation afford a unique opportunity to investigate the intrinsic mechanisms responsible for BCL-2 dependence and identify biology that can be harnessed to increase venetoclax sensitivity in the broader resistant MM population. Experiments were performed to determine whether metabolism and cellular energetics play a role in the increased BCL-2 dependence and sensitivity of t(11;14) MM to venetoclax.

The primary translocations in myeloma occur early in the evolution of the disease and the t(11;14) translocation juxtaposes the cyclin D1 gene to the immunoglobulin heavy chain (IgH) gene enhancer resulting in dysregulated cyclin D1 expression. Cyclin D1-dependent activation of CDK4/6 promotes G1-S phase cell cycle transition. Cyclin D1 also exhibits a number of CDK independent functions by regulating more than 35 transcription factors to block cellular differentiation, proliferation, mitochondrial biogenesis and mitochondrial oxidative phosphorylation. While data analysis does not have matching venetoclax sensitivity data making it difficult to identify a venetoclax sensitive gene signature, the observation of reduced ETC gene expression in t(11;14) vs. non-t(11;14) indicates that ETC activity may be a biomarker of venetoclax sensitivity. The t(11;14) translocation was used as a proxy as ˜40% of t(11;14) patients are venetoclax sensitive whereas <6% of non-t(11;14) are venetoclax-sensitive. This is supported by our functional studies in cell lines that demonstrated reduced ETC activity to be important to venetoclax sensitivity. Low SQR activity is a reliable read-out of venetoclax sensitivity in all MM cells, irrespective of t(11;14) status. Importantly, SQR activity can be evaluated in intact permeabilized whole cells in a fast and direct activity measurement assay. Direct assessment of the ubiquinone reductase activity of Complex I is technically challenging as it requires isolation of mitochondria followed by permeabilization while maintaining the structure and activity of Complex I. SQR activity on the other hand can be determined in semi-permeabilized whole cells supplemented with exogenous succinate.

Experiments were also performed to determine whether an elevation in OXPHOS promotes resistance to venetoclax. Glutamine deprivation (that suppresses OXPHOS) sensitizes resistant MM cell lines and patient samples to venetoclax. Sensitization to venetoclax was reversed upon supplementation of the glutamine-deprived cells with cell permeant a-ketoglutarate indicating that restoration of OXPHOS promotes resistance to venetoclax. Interestingly, experiments in venetoclax-relapsed CLL also demonstrate changes in cellular metabolism and an elevation in OXPHOS to promote venetoclax resistance, supporting observations of elevated OXPHOS correlating with reduced BCL-2 dependence. The dependence of B cells on specific prosurvival BCL-2 members is activation, development and differentiation stage specific.

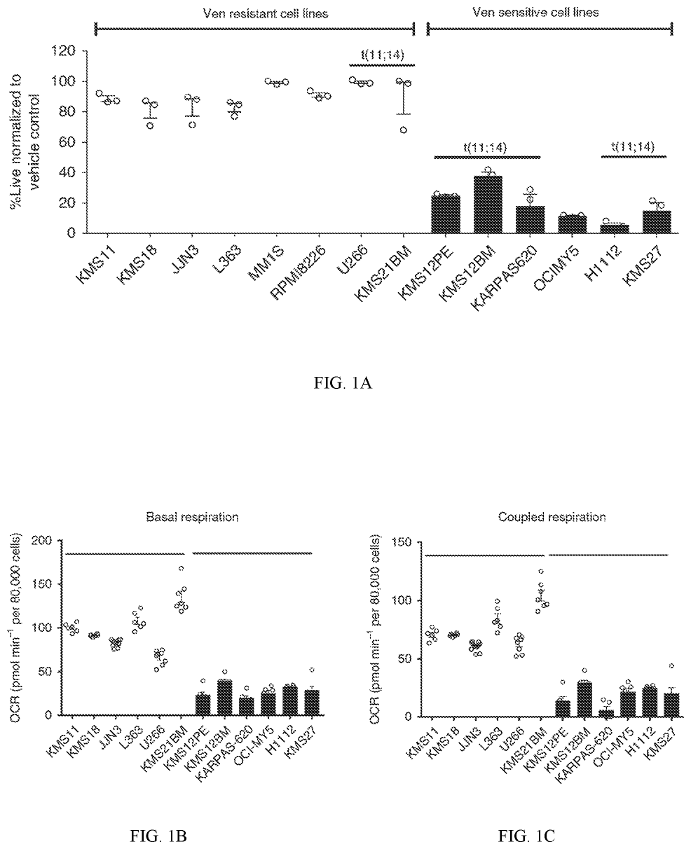

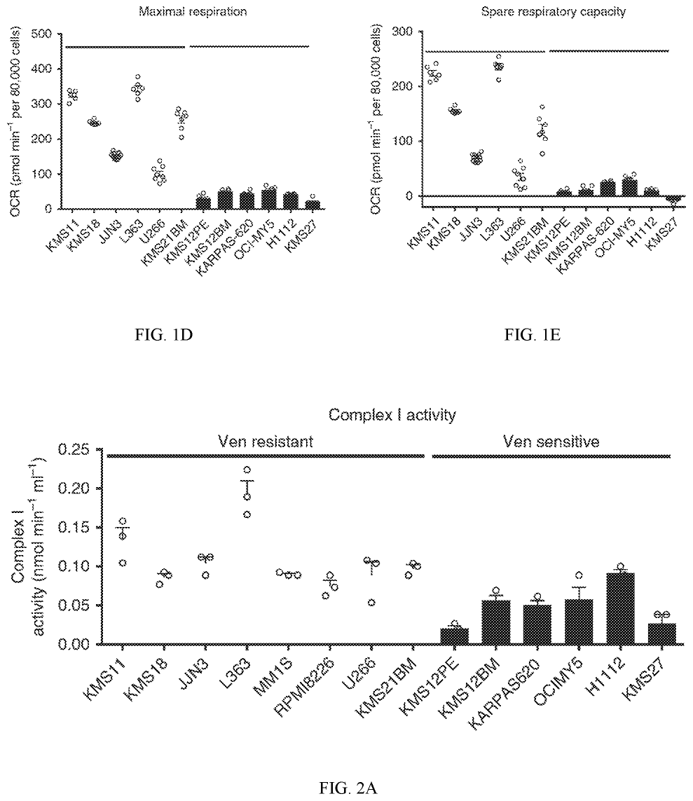

The activation/differentiation of normal murine B cells is associated with increased ABT-737 (a Bcl-2, Bcl-xL, and Bcl-w antagonist) sensitivity due to a decline in BCL-2 and increase in BCL-xL expression upon differentiation to a plasma cell. Studies testing the effects of ABT-737 on the murine immune system indicate that ABT-737 inhibits the establishment of newly arising bone marrow plasma cells and memory B cells with no effect on pre-existing plasma cells, or germinal center B cells. These results indicate that in B cells, BCL-2 dependence is regulated in an activation and/or differentiation stage-specific manner. With respect to metabolism, B-cell activation and differentiation to a plasma cell are associated with an increased dependency on the TCA and OXPHOS. t(11;14) MM are differentiated antibody-producing plasma cells. However, studies examining expression profiles of CD138+MM in the CD2 subgroup of CCND1/CCND2-amplified MM cells identified elevated expression of B-cell lineage markers like MS4A1/CD20, VPREB and PAXS. Thus, a multiple myeloma plasma cell malignancy which should be exhibiting elevated OXPHOS may get locked into metabolically compromised suppressed ETC state upon expressing/maintaining B-cell-like features. This suppression of the ETC activity is thus also associated with the sensitivity to venetoclax (FIG. 1A), allowing for a potent strategy of synthetic lethality with venetoclax. Interestingly, the non-t(11;14) OCI-MY5 line that exhibits low SQR activity and venetoclax sensitivity is also likely to be more B-cell-like or undifferentiated based on its high expression of CD20. SQR activity in the limited number of DLBCL lines assessed was low. A correlation between low SQR activity and venetoclax sensitivity was not detected supportive of a differentiation specific component to ETC activity and BCL-2 dependence.

Experiments reported herein indicate that the ETC is a target for venetoclax sensitization. Initial studies focused on Complex II. SDH is unique in that it is the only ETC complex with all four subunits being solely nuclear encoded and exclusively positioned to function in both the TCA and ETC. The four SDH subunits have distinct roles with mutations in SDHA linked to impaired oxidative phosphorylation and severe metabolic disorders while mutations in SDH B, C and D are linked to tumorigenesis when present with Complex I deficiency. Disruption of SQR activity with the introduction of the SDHC R72C mutant or with TTFA treatment that disrupts the SQR activity of the enzyme without impacting SDH activity or Complex I activity sensitizes MM cells to venetoclax, underscoring the sufficiency of targeting SQR to induce BCL-2 dependence. Death-resistant cells have increased SRC and Complex II plays a role in maintaining SRC and resistance to death. Venetoclax-resistant MINI exhibit significantly higher SRC than venetoclax-sensitive MM and inhibition of SQR by TTFA or the R72 SDHC mutant lowers the SRC of venetoclax-resistant MM and sensitizes them to venetoclax. Selective potent SQR inhibitors create a heightened dependence on BCL-2 and venetoclax sensitivity. Studies in AML have shown that targeting Complex II with azacitidine induced venetoclax sensitivity. The oncometabolite (R)-2-HG expressed in IDH 1/2 mutant-expressing AML induces BCL-2 dependence by suppressing cytochrome c oxidase (Complex IV). Targeting the other ETC complexes can also sensitize to venetoclax. The unique aspect of Complex II is not in contributing to proton transport but being a conduit of electrons from succinate to labile ubiquinone, whose inhibition can generate signals that can elevate BCL-2 dependence.

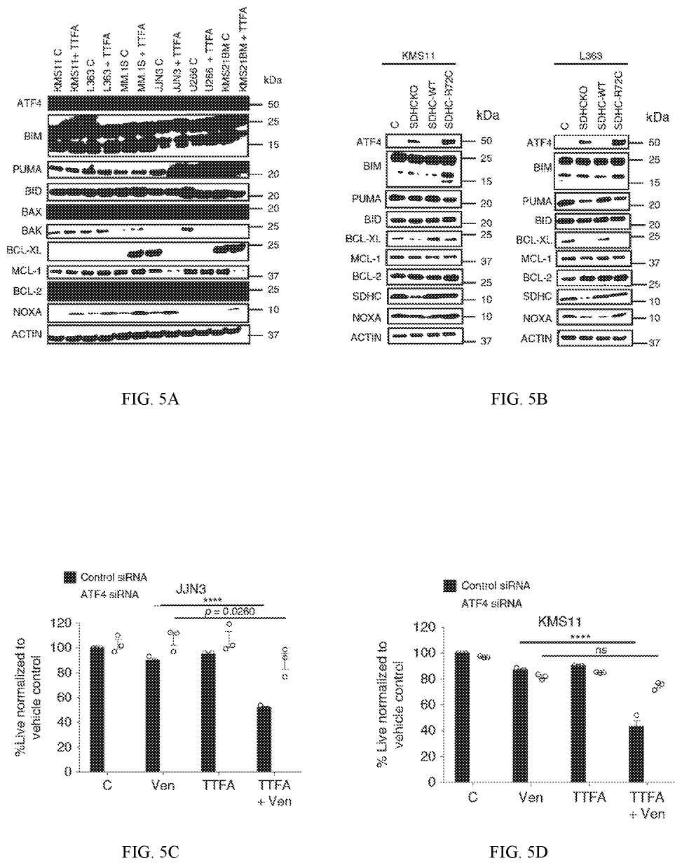

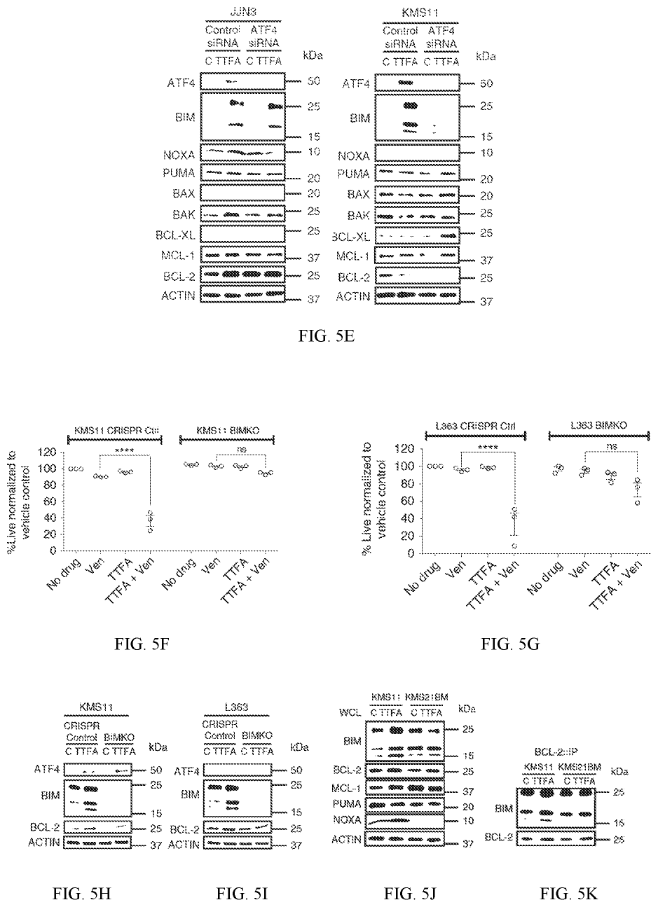

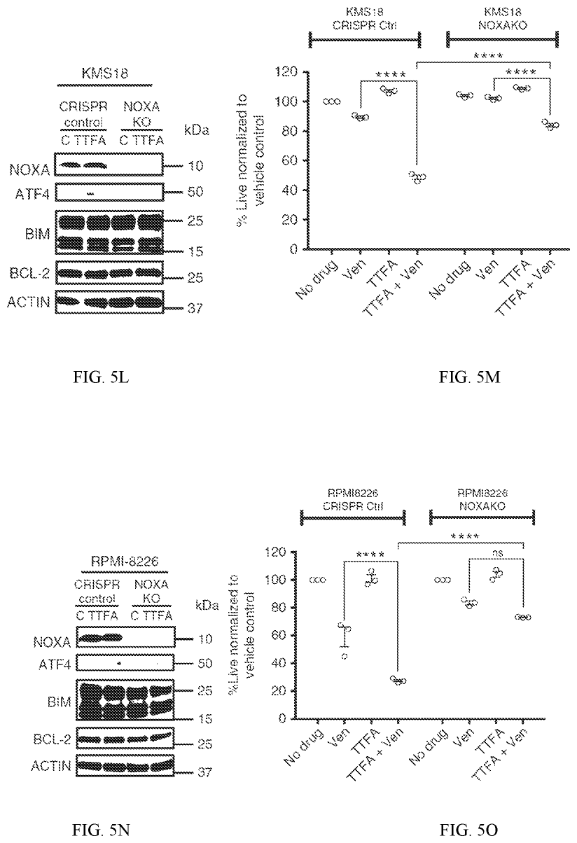

Complex II is linked to the generation of ROS, accumulation of succinate and the induction of HIF1-α that can lead to induction of ATF4. ATF4 is post-translationally induced as part of the integrated stress response to anoxia, hypoxia, proteasome inhibition or nutrient deprivation, upregulating genes that sustain amino acid metabolism and anti-oxidant responses. ATF4 has both prosurvival effects through the activation of autophagy and proapoptotic effects through the induction of C/EBP homologous protein (CHOP) which in turn induces BIM, NOXA and PUMA. NOXA is implicated in inducing BCL-2 dependence and sensitivity to venetoclax, implicated in bortezomib cytotoxicity in MM and in panobinostat sensitivity of DLBCL. ATF4 in specific cellular contexts can induce NOXA which, in binding MCL-1, can increase the quantity of BIM bound to BCL-2, potentially explaining TTFA induced BCL-2 dependency. It is noted that U266 cells do not express basal NOXA, and NOXA was not induced with TTFA treatment in U266 which could contribute to its reduced sensitivity to BCL-2 antagonists. However, interestingly, while both ATF4 and NOXA are induced in KMS21BM upon treatment with TTFA and IACS-010759, 100 μM TTFA does not sensitize these cells to venetoclax. This can be explained by the difference in the levels of induction of ATF4 and NOXA proteins upon Complex I and Complex II inhibition. Concordantly, the higher level of ATF4 and NOXA induction upon IACS-010759 treatment is enough to sensitize KMS21BM to venetoclax. On the other hand, the low level of NOXA induction in case of TTFA treatment of KMS21BM is not sufficient to increase BIM binding on BCL-2 (FIG. 5K) and sensitize the cells to venetoclax treatment.

Refractory cancers, including MM and other cancers subject to prior chemotherapy, exhibit reduced “priming” i.e. exhibit suboptimal quantities of BH3 activators bound to antiapoptotics, increasing the threshold required to induce apoptosis. Hence, approaches that can increase the “primed” state of the cell will not only increase sensitivity to existing chemotherapy but also enhance sensitivity to BH3 mimetics. Normal cells, on the other hand, being further removed from the apoptotic threshold are not sensitized by ETC inhibition and venetoclax cotreatment. It is the presence of minimal residual disease (MRD) that is well connected to the emergence of refractory disease. There continues to be an unmet need in achieving deeper responses in t(11;14) patients to remove MRD and reduce toxicity. Therefore, identification of novel targets to achieve sensitization even in t(11;14) patient samples is important. The isolated myeloma patient samples, in contrast to cell lines, are not proliferating ex vivo. It is possible that proliferating cells (more characteristic of poor prognosis MM) are more dependent upon OXPHOS due to energetic and biosynthetic requirements of cell division and thus could make them more sensitive to targeting ETC activity. Given the genetic heterogeneity of MM, targeting the ETC has the potential to maximize the clinical application of the highly potent BCL-2 antagonist, venetoclax, and provide a more unifying approach to tackle refractory resistant MM.

Myeloma exhibit reduced Complex I mutations compared to acute myeloid leukemia (AML) and could potentially respond better to Complex I inhibition.

### Venetoclax-Sensitive MM Exhibits Reduced Cellular Energetics in Contrast to Venetoclax-Resistant MM.

Differential venetoclax sensitivity was assessed in a panel of MM lines. Venetoclax elicited significant cytotoxicity primarily in t(11;14) lines (FIG. 1A). The pattern of increased sensitivity of these cell lines was selective for venetoclax and not detected with other standard myeloma therapeutics i.e. bortezomib and melphalan or sensitivity to the MCL-1 inhibitor 563845 as reported for the KMS12BM, KMS12PE, KMS11, MM.1 S, JJN3, RPMI-8226 and L363 lines.

To investigate differential energy metabolism in venetoclax sensitive and resistant cells, glucose and glutamine carbon isotope tracing was performed using labeled U¹³C-glucose or U¹³Cglutamine in resistant non-t(11;14) KMS11 and t(11;14) U266, as well as sensitive t(11;14) KMS12PE and non-t(11;14) OCI-MY5 cell lines. Lower TCA cycle metabolite levels were detected in the sensitive compared to venetoclax-resistant lines. Glucose-derived carbon contribution to the TCA cycle intermediates was reduced in venetoclax-sensitive cells, reflected in the diminished pool of citrate, succinate, fumarate and malate along with decrease in ¹³C enrichment of citrate, a-ketoglutarate, succinate, fumarate and malate in cells supplemented with U-¹³C-glucose. In contrast, comparable glutamine derived carbon utilization was detected. The results cannot be explained by reduced nutrient uptake or reduced mitochondrial content in the venetoclax-sensitive lines as these cells do not exhibit a uniformly reduced pattern of glucose or glutamine consumption or proliferation rate, and mitochondrial mass as assessed by cardiolipin staining.

Next, oxygen consumption rates (OCR) were evaluated in the MM cell lines. Basal, maximal, coupled OCR and spare respiratory capacity (SRC) were found to be significantly lower in all of the sensitive MM lines (FIG. 1B-1E). Interestingly, insensitive t(11;14) lines such as U266 and KMS21BM displayed high basal, maximal and coupled respiration while the sensitive line, OCI-MYS, which does not have a 11;14 translocation, exhibited lower respiratory parameters suggesting that oxygen consumption and potentially electron transport chain (ETC) activity could segregate venetoclax-sensitive and -resistant MM cells. Importantly, SRC, which is the difference in basal and maximal respiration and reflects the potential to elevate cellular bioenergetics and ATP synthesis during oxidative stress or upon partial Complex I inhibition, is also reduced in the sensitive cells. In sum, these results suggest that the ETC activities may be suppressed in venetoclax-sensitive cells.

To discern the basis for differential OCR, the CoMMpass MM trial (NCT0145429, IA11) RNAseq data was reviewed to examine ETC gene expression. As the response of these patients to venetoclax is unknown, t(11;14) was used as a proxy for venetoclax sensitivity. Interestingly, this analysis demonstrated marked suppression of ETC-related genes in patients with t(11;14) MM which is in line with the OCR assessment of t(11;14) and venetoclax-sensitive cell lines.

### Venetoclax-Sensitive MM Exhibits Reduced Complex I and Complex II Activity.

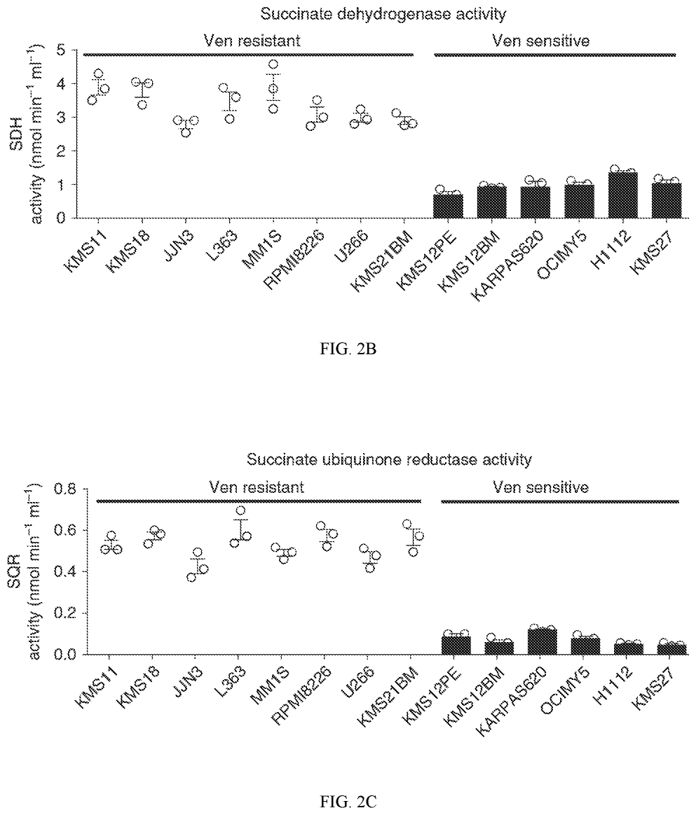

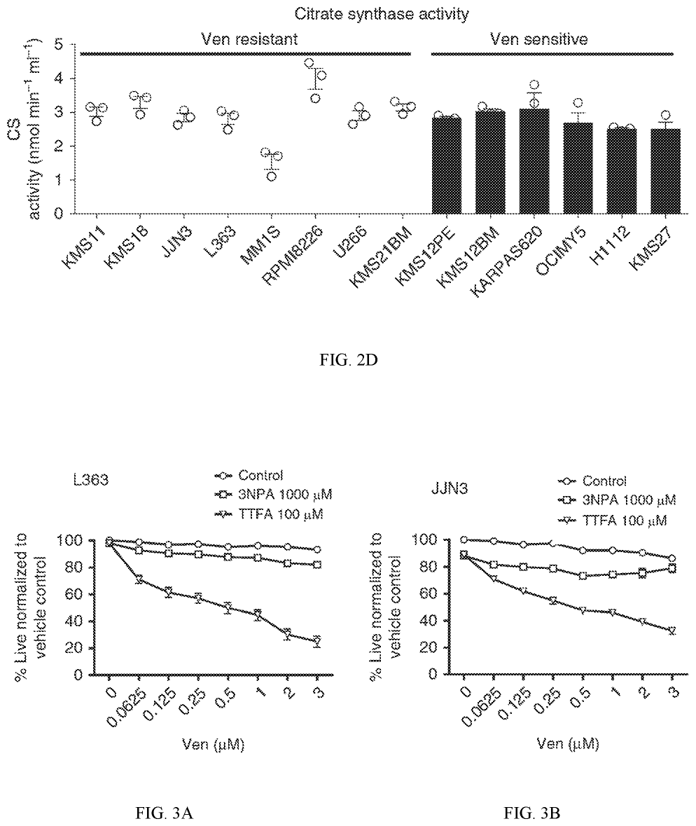

Given reduced oxygen consumption rates (OCR) in sensitive cells, the activities of mitochondrial Complex I and II were examined. Mitochondrial Complex I and II receive electrons from the electron donors NADH or FADH2 respectively, that are transferred to the terminal electron acceptor (O₂) via a series of redox reactions. Complex I or NADH:Coenzyme Q:oxidoreductase is the largest multimeric respiratory complex of ETC comprised of over 40 subunits with its catalytic subunit containing the NADH binding site. Electrons from NADH are transferred to FMN and then to FeS clusters and finally to ubiquinone where ubiquinone is reduced to ubiquinol. Complex II is the only enzyme that has a direct role both in the TCA cycle and ETC. Complex II/Succinate dehydrogenase (SDH) consists of four subunits: SDHA, SDHB, SDHC and SDHD that contain distinct dehydrogenase and oxidoreductase enzymatic activities. SDHA exposed to the mitochondrial matrix contains the catalytic dicarboxylate binding site for succinate. SDHA is responsible for the SDH activity of Complex II and oxidizes succinate to fumarate in addition to reducing FAD to FADH2. Electrons from FADH2 are then transferred sequentially to Fe—S clusters in SDHB and then to ubiquinone at the Qp site formed by SDHC and D, embedded in the mitochondrial inner membrane. The reduction of ubiquinone bound in the Qp site to ubiquinol is referred to as the succinate ubiquinone reductase (SQR) activity of Complex II. Complex I activity was measured using an assay that relies on immunocapture of Complex I from freshly prepared cellular protein extracts. The NADH dehydrogenase activity of immunocaptured Complex I was determined by the oxidation of NADH and simultaneous reduction of the provided dye resulting in increase in absorbance. SDH and SQR activities were measured in live permeabilized cells, containing intact mitochondria pretreated with inhibitors of Complex I (rotenone), Complex III (antimycin) and Complex IV (sodium azide), allowing for selective measurement of SQR or SDH activity. In the assay, SDH activity correlates with electron transfer from succinate to the Fe-S clusters to the water-soluble dye 5-methyl-phenazinium methyl sulfate (PMS) to the final acceptor 3-(4,5-dimethylthiazol-2-yl)-2,5-diphenyltetrazolium bromide (MTT). On the other hand, SQR activity correlates with electron transfer from succinate to the Fe-S clusters to decylubiquinone bound at the Qp site and then final transfer to the redox sensitive dye, 2,6-dichlorophenolindophenol (DCPIP). The assay is validated by detection of SDH activity only upon addition of the electron donor i.e. succinate and inhibition of activity upon addition of malonate (dicarboxylate binding site competitor) or selective SQR inhibition with thenoyltrifluoroacetone (TTFA). Venetoclax-sensitive cells exhibit significantly lower Complex I activity and SQR and SDH activities than resistant lines including t(11;14) U266 and KMS21BM, correlating with venetoclax sensitivity. The activity of citrate synthase (a TCA cycle enzyme) was similar in sensitive and resistant lines (FIG. 2D) suggesting the presence of an equivalently functional TCA cycle enzyme activity in contrast to the ETC activities. Interestingly, Complex I subunit (NDUFS2) and SDH subunit protein expression levels did not correlate with Complex I, SDH or SQR activity (FIG. 2A-2C), underscoring the importance of assessing enzymatic activity over subunit gene expression to explain their functional contribution.

Experiments were performed to determine whether other cancers demonstrate a correlation between low SQR activity and venetoclax sensitivity. DLBCL lines ((SUDHL-4, SUDHL-6, U2932, OCILY19, OCI-LY10, HBL-1) were evaluated for both SQR activity and venetoclax sensitivity. A correlation between low SQR activity and venetoclax sensitivity was not detect suggesting that the relationship between low SQR activity and BCL-2 dependence may be plasma cell context specific.

Inhibition of SQR with Qp Site Inhibitor TTFA Sensitizes Resistant MM to Venetoclax

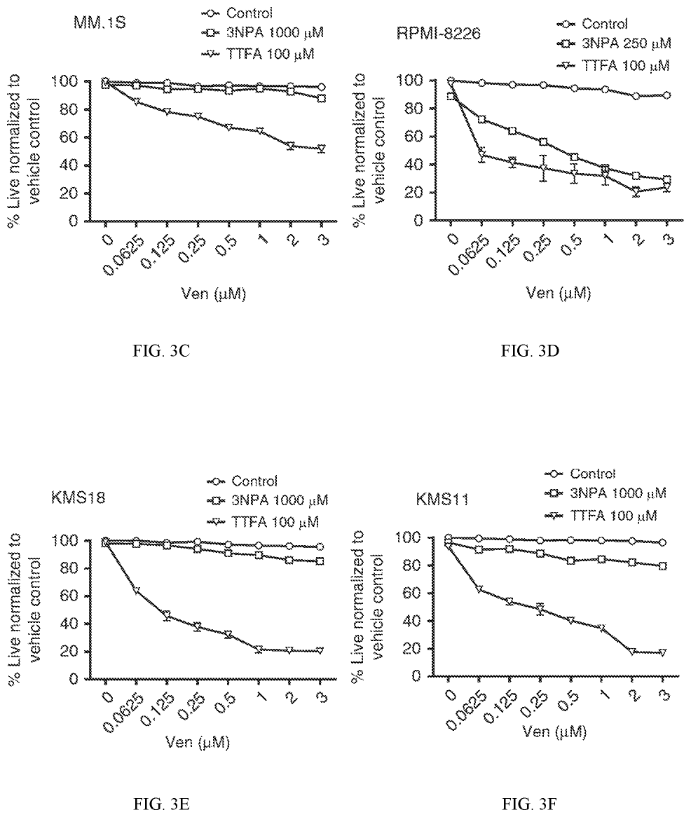

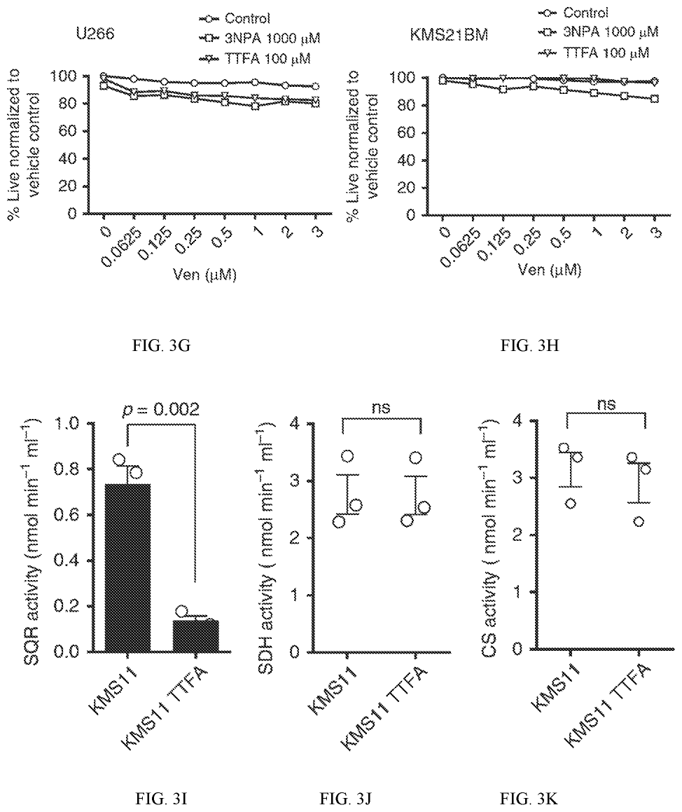

Given the unique role of Complex II in directly connecting the TCA cycle to the ETC, the impact of selectively inhibiting either the SDH or SQR activities of Complex II on venetoclax sensitivity was tested. The venetoclax sensitization effects of TTFA (SQR inhibitor) or 3NPA (SDH inhibitor) were tested on eight venetoclax-resistant MM lines (FIG. 3A-3H). 3NPA is a structural analog of succinate, which binds irreversibly with SDH inhibiting FAD reduction and fumarate production while TTFA is a direct Qp site inhibitor. Inhibition of SQR in resistant MM lines, with the Qp site inhibitor TTFA, increased sensitivity to venetoclax in six of the eight lines tested. 3NPA on the other hand was less effective compared to TTFA in eliciting sensitivity to venetoclax. The efficacy of the TTFA-venetoclax cotreatment strategy was also tested in a colony-forming unit assay. MM lines (L363 and KMS11) were cultured in an agar matrix with single agents or the combination. Similar to results obtained in suspension culture, TTFA treatment sensitized MM lines to a lower dose of venetoclax (0.1 μM) reducing cellular viability. These results highlight the sufficiency of targeting the Qp site/quinone reductase activity of Complex II to induce BCL-2 dependence and sensitization to venetoclax. To demonstrate the selectivity of TTFA inhibiting SQR activity, SQR, SDH, and CS activities were evaluated in TTFA-treated KMS11 cells. Significant reduction in SQR activity was found upon TTFA treatment (FIG. 31), while SDH (FIG. 3J) and CS (FIG. 3K) activities remained unaffected. SRC was also concordantly reduced in the TTFA treated KMS11 cells but not in unsensitized U266 and KMS21BM cells.

### SQR Inhibition Via Qp Site SDHC-Mutant Introduction is Sufficient to Sensitize MM to Venetoclax

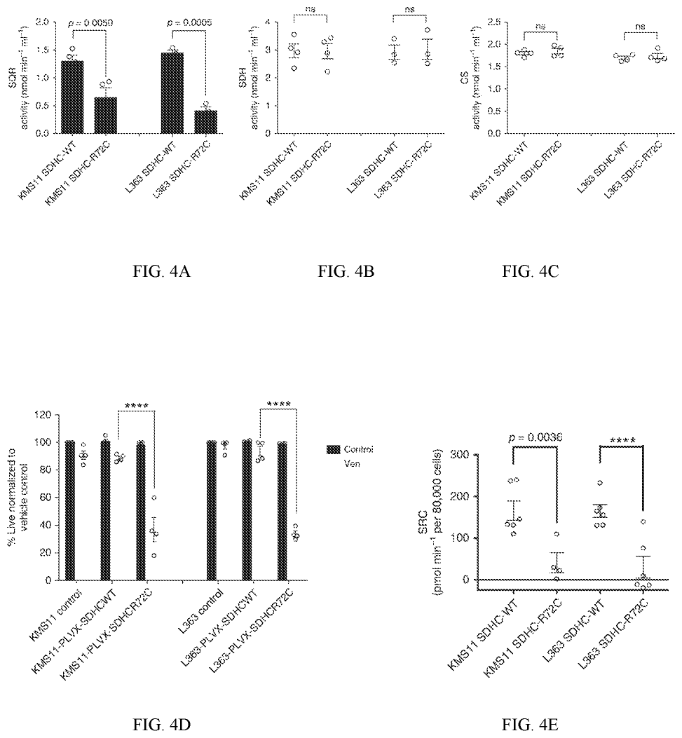

To further interrogate the sufficiency of SQR inhibition in inducing venetoclax sensitivity, a Qp site variant of SDHC was generated by mutating arginine to cysteine at position 72 which shows less binding affinity with ubiquinone. An SDHC guide RNA was generated to target an intronic region prior to the first SDHC exon. The mutant was expressed in SDHC knockout (KO) L363 and KMS11 cell lines. This strategy was adapted as the SDHC KO cells did not survive in culture. Cells expressing the SDHC-R72C mutant exhibited selective reduction of SQR activity (FIG. 4A) while maintaining SDH (FIG. 4B) and CS (FIG. 4C) activity as compared to SDHC-WT cells. Complex I activity was maintained in SDHC-R72C mutant-expressing KMS11 cells. The SDHC-R72C mutant sensitized KMS11- and L363-resistant MM cells to venetoclax (FIG. 4D), demonstrating that the Qp site and SQR inhibition is sufficient for inducing venetoclax sensitivity, despite maintenance of SDH, CS and Complex I activities. Since venetoclax-sensitive cells exhibited reduced mitochondrial respiratory parameters, basal, maximal and coupled respiration were evaluated in parental and SDHC-R72C mutant cells. Both KMS11 and L363 SDHC-R72C mutant cells exhibited a reduction in SRC (FIG. 4E), concordant with the loss of SQR activity. In summary, these results demonstrate that selective Complex II SQR inhibition increases BCL-2 dependence and venetoclax sensitivity.

### ATF4, BIM and NOXA Regulate TTFA-Induced Venetoclax Sensitivity in MM

To identify the mechanistic basis for SQR inhibition-related venetoclax sensitivity, the expression of ATF4, a transcription factor typically upregulated in response to cellular stresses such as ER stress, hypoxia and amino acid deprivation, was evaluated. expression levels and binding properties of BCL-2 family members in SDHC-R72C mutants and resistant MM cells treated with and without TTFA were also evaluated. Increased expression of ATF4, NOXA, and a variable increase in BIM and/or BCL-2 protein expression in TTFA treated MM cells were detected (FIG. 5A). In addition, SDHC-R72C expressing mutants (FIG. 5B) exhibited a similar pattern of ATF4, BIM and BCL-2 induction. To interrogate the significance of ATF4 induction in venetoclax sensitization, ATF4 expression in KMS11 and JJN3 cells was suppressed using an ATF4 siRNA pool. Knockdown (KD) efficiency demonstrated in (FIG. 5E). Control and ATF4 siRNA-transfected cells were treated with or without TTFA and assessed for sensitivity to venetoclax. ATF4 KD significantly reversed venetoclax sensitivity, rescuing cell viability in venetoclax and TTFA cotreated cells (FIG. 5C and 5D) suggesting its role in elevating BCL-2 dependence. Importantly, TTFA-induced NOXA expression was reversed with ATF4 KD (FIG. 5E), while the induction of BIM and BCL-2 was variable. BIM induction was reduced in TTFA-treated KMS11 cells with ATF4 KD but maintained in TTFA-treated JJN3 cells (FIG. 5E).

Knockdown of ATF4 also reduced the induction of BCL-2 and BAK in TTFA-treated JJN3 cells, identifying cell context-specific contributory roles of other BCL-2 family proteins in elevating BCL-2 dependence in SQR-inhibited cells. The reduction in NOXA induction detected in the ATF4 KD cells (KMS11 and JJN3) treated with TTFA (FIG. 5E) suggests a role for NOXA in TTFA-induced venetoclax sensitivity. NOXA can displace BIM bound to MCL-1 to promote BIM binding to BCL-2 thereby increasing BCL-2 dependence in MM.

To evaluate the role of BIM in TTFA-induced BCL-2 dependency, a CRISPR/Cas9 knockout of BIM was generated in KMS11 and L363 cells. BIM KO reversed the cytotoxic effect of cotreatment with TTFA and venetoclax in both KMS11 and L363 cells (FIG. 5F, 5G). This effect was observed despite the maintenance of ATF4 expression (FIG. 5H, 5I), suggesting it to be a downstream effector. These results confirm BIM as the major proapoptotic protein in TTFA/SQR inhibition-induced venetoclax sensitivity. The requirement for NOXA induction in TTFA-induced venetoclax sensitivity was also tested. Using NOXA KO lines (RPMI-8226 and KMS18), a lack of induction of NOXA in TTFA-treated NOXA KO was demonstrated (FIG. 5L, 5N), and, importantly, a loss of TTFA-induced sensitivity to venetoclax (FIG. 5M, 5O). NOXA KO efficiency and expression levels of ATF4, BIM and BCL-2 were evaluated and are shown in (FIG. 5L, 5N).

The regulation of ATF4, BIM and NOXA in MM cells that were not sensitized by TTFA treatment were investigated. Whole-cell lysates were evaluated for total protein expression. Coimmunoprecipitations was performed to examine binding of BIM with antiapoptotic BCL-2 in KMS11 and the TTFA insensitive-t(11;14) KMS21BM cells. Induction of BIM and NOXA were observed upon TTFA treatment (FIG. 5J) only in KMS11 not in the TTFA insensitive-t(11;14) KMS21BM cells. Coimmunoprecipation of BCL-2 from control or TTFA-treated cells also demonstrated elevated BIM binding to BCL-2 in TTFA-treated KMS11 cells in contrast to the resistant KMS21BM cells (FIG. 5K). Cumulatively, these results identify NOXA and BIM to facilitate TTFA-ATF4-induced venetoclax MM sensitization.

SQR Activity Inversely Correlates with Venetoclax Sensitivity in MM Patient Samples

MM patient samples were used to (1) evaluate the impact of TTFA treatment on inducing sensitivity to venetoclax and (2) evaluate SQR activity in purified MM cells and its correlation with venetoclax cytotoxicity ex vivo and the response of patients being administered venetoclax (Trial NCT01794520: a Phase I Study Evaluating the Safety and Pharmacokinetics of venetoclax in Subjects with Relapsed or Refractory Multiple Myeloma). Complex I comprising of over 40 subunits is difficult to assay. A direct evaluation of the NADH-ubiquinone oxidoreductase activity of Complex I requires release of Complex I from whole cells or manipulation of purified mitochondria as mitochondria are impermeable to NADH. On the other hand, SQR activity can be assessed in a fast and direct activity measurement assay using permeabilized cells. Due to the caveats associated with Complex I activity measurement and the simplicity of the SQR activity measurement assay, SQR activity was assessed in purified CD138+ MM cells from patient samples.

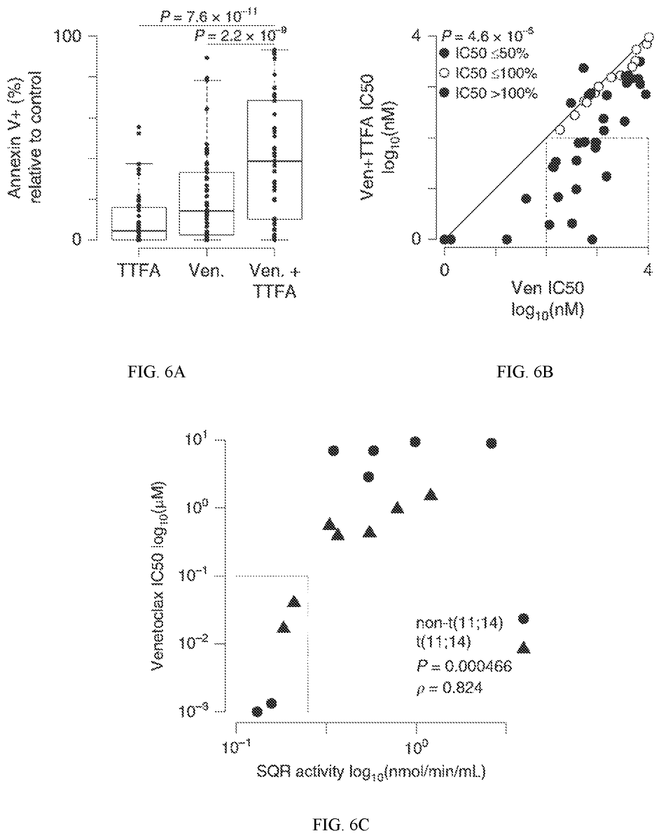

Bone marrow aspirate samples from 50 MM patients were treated with three doses of venetoclax (0.01, 0.1 and 0.5 μM) or TTFA (100 μM), or the combination for 24 h and cell death was assessed by AnnexinV staining. These results show that the combination of TTFA plus 0.1 μM venetoclax resulted in significantly more cell death in 50 primary myeloma cells (FIG. 6A). A venetoclax ICso <0.1 μM is characterized as a sensitive sample. By these criteria 15 of 50 (30%) samples were sensitized by TTFA treatment. However, comparison of the ICso of venetoclax plus TTFA vs.venetoclax alone showed that 46 of 50 (92%) had increased venetoclax sensitivity with 31 (62%) showing more than a 50% decrease in IC50 (FIG. 6B). Of the 31 samples where TTFA did reduce their IC₅₀ by at least 50%, 11 exhibited the t(11;14) translocation. TTFA sensitized MM cells to venetoclax, increasing cell death in CD38+ CD45− gated MM cells with no to minimal impact on viability of the non-MM cells contained within the aspirate, suggestive of the selective synthetic lethality of this combination in MM cells.

SQR activity was assessed in purified CD138+MM cells from 14 samples. There was a positive correlation between SQR activity and venetoclax insensitivity/resistance ex vivo, validating observations in cell lines where low SQR activity correlates with venetoclax sensitivity (FIG. 6C). Low SQR activity (i.e. <0.25 nmol min⁻¹ mL⁻¹) was identified in four patient samples, including two non-t(11;14), to correspond with ex vivo venetoclax sensitivity i.e. venetoclax ICso <0.1 μM. In addition, three patients which were t(11;14), a non-t(11;14), either on the M15-538 trial or off trial were found to have low SQR activity and a clinical response.

On the other hand, among the ten resistant samples (based on ex vivo sensitivity), all having high SQR activity, five exhibited the t(11;14) translocation, highlighting the ability of SQR activity to identify venetoclax-resistant MM among t(11;14) MM. One of these patients was a post-venetoclax treatment refractory sample, the other failed to achieve an objective response and the third was responsive. While all ten resistant samples showed increased sensitivity to venetoclax upon TTFA treatment, seven samples showed greater than 50% reduction in ICso values. Importantly, one that showed more than tenfold reduction in ICso was the t(11;14) patient who relapsed on venetoclax and was additionally refractory to other therapies. This patient's MM cells exhibited high SQR activity highlighting (1) SQR activity correlates with a refractory patient response and the ability of SQR inhibition to induce venetoclax sensitization.

### Inhibition of Complex I and Distal ETC Complexes Sensitize MM to Venetoclax

To further inquire whether Complex I inhibition could also sensitize MM cells to venetoclax, a Complex I inhibitor was tested (IACS-01075938). Treatment of three representative resistant lines (KMS11, L363 both non-t(11;14); and KMS21BM a t(11;14) line) with increasing doses of IACS-010759 sensitized cells to 0.5 μM venetoclax after a 24 h treatment. Further, Complex I inhibitor piericidin also sensitized resistant MM cell lines to venetoclax. Western blot analysis of cells treated with 25 nM IACS-010759 for 24 h revealed induction of ATF4 and NOXA suggesting that, similar to TTFA, IACS-010759 also sensitizes MINI cells to venetoclax through regulation of ATF4 and NOXA. The dose of IACS-010759 was selected based on proximity to the ICso of IACS-010759 in combination with 0.5 μM venetoclax and minimal impact of single-agent IACS-010759 on viability after a 24 h treatment. Cotreatment with 25 nM IACS-010759 significantly reduced the IC50 of venetoclax in the tested cell lines.

Experiments were performed to determine whether inhibition of Complex III, IV and V could also sensitize resistant MINI to venetoclax. As seen with Complex I and SQR inhibition, inhibition of Complex III, IV and V by Complex III inhibitor (antimycin), Complex IV inhibitor (sodium azide) and Complex V inhibitor (oligomycin) at doses which reduced ATP levels by about 50% after 24 h sensitized L363, KMS11 and KMS21BM to venetoclax

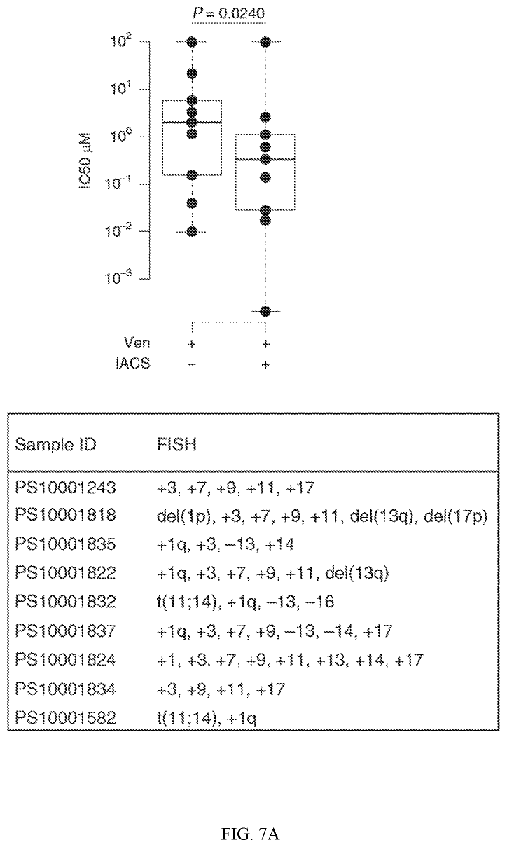

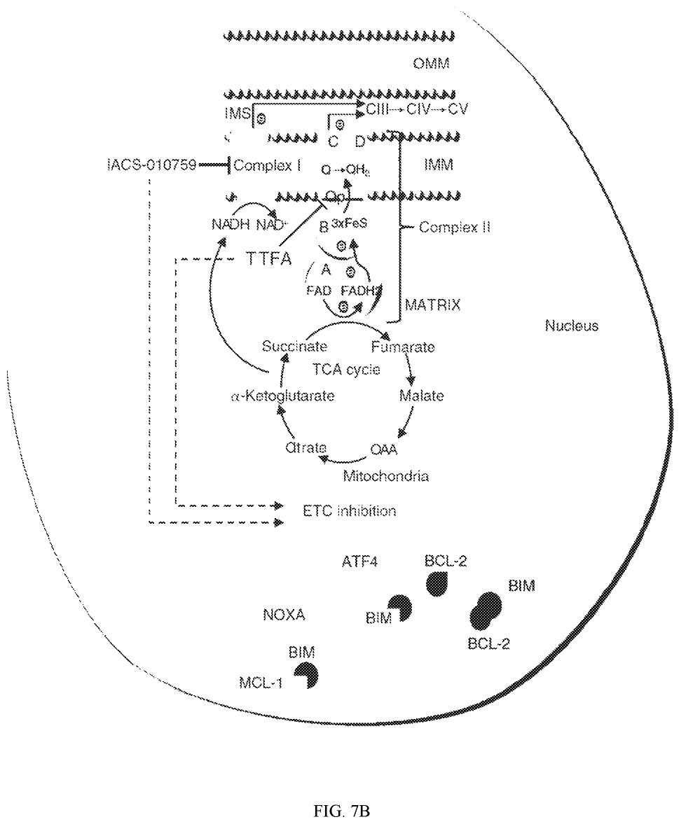

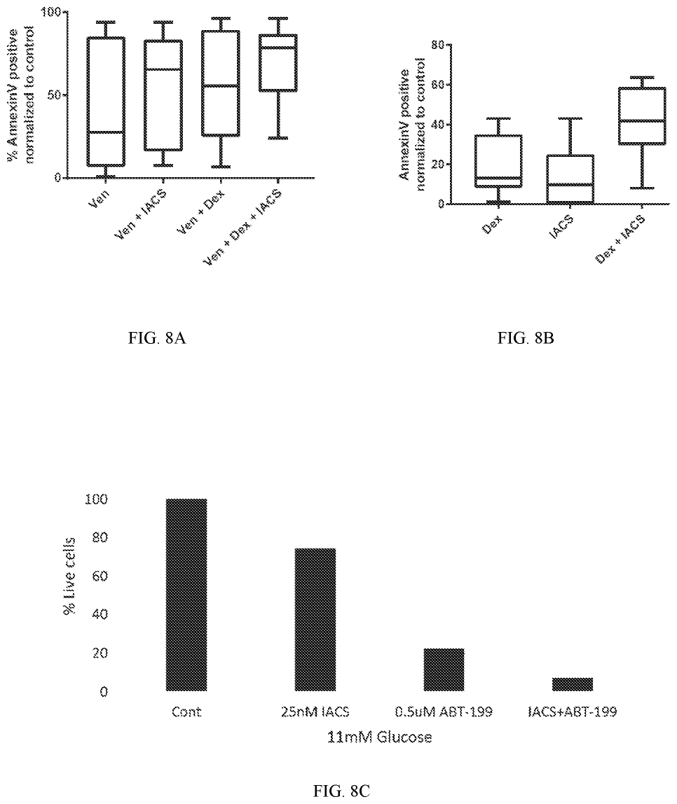

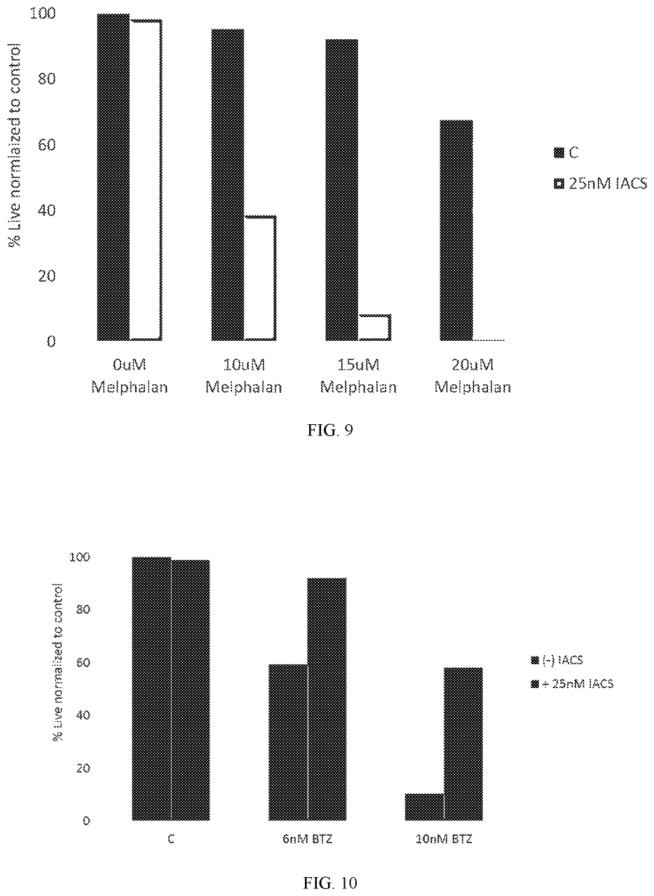

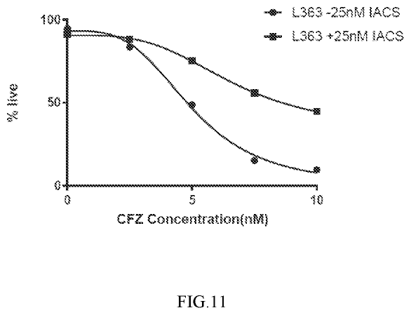

The efficacy of IACS-010759 in sensitizing was tested in MINI patient samples to venetoclax. Eight out of nine samples showed increased sensitivity with IACS-010759 and there was >50% reduction in the ICso of venetoclax in five of the samples. Interestingly, out of the two t(11;14) patient samples, one was resistant to venetoclax alone and was significantly sensitized on treatment with IACS-010759 (IC50 of Ven+IACS=0.01732 μM). This suggests that IACS-010759 can be used to sensitize non-t(11;14) MM and venetoclax-resistant t(11;14) patients, only 40% of which are responsive to single-agent venetoclax. These results are summarized in FIG. 7A. Additionally, similar to TTFA, combined treatment of IACS-010759 with venetoclax had minimal impact on the viability of non-MM cells contained within the aspirate. In sum, IACS-010759 reduced the IC₅₀ for venetoclax and sensitized MINI patient samples to single-agent venetoclax.

### Isolation and Purification of Primary Myeloma Cells

Bone marrow aspirates or peripheral blood samples from consenting myeloma patients were diluted to 25 mL with PBS and over laid on lymphocyte separation media. Following centrifugation, the collected buffy coat was washed with PBS and resuspended in culture medium. Cells subject to various treatments were stained with anti-CD38-phyocerythrin and anti-CD45-allophycocyanin-Cy7 to identify MM cells. CD138+ cells were purified using MACS CD138+ human microbeads. IC_{50S} of venetoclax alone or in combination with TTFA/IACS-010759 in myeloma samples were calculated using nonlinear regression (curve-fit) analysis under agonist vs. normalized response with variable slope.

### Enzyme Activity Assays

Succinate ubiquinone reductase (SQR) and succinate dehydrogenase (SDH) activity assays were performed by absorbance-based assays using different electron acceptors. 0.3×10⁶ cells were harvested per sample, washed with PBS and resuspended in buffer containing 10 mM KH₂PO₄ (pH 7.4), 2 mM EDTA and 1 mg per mL BSA, 10 mM sodium azide, 5 μM rotenone, and 2 μM antimycin (assay buffer). Control, 50 μM TTFA and 5 mM malonate treated samples were supplemented with 20 mM succinate. Sample without succinate was used as a negative control for the assay. For SQR assay, reaction was initiated by adding reaction buffer containing 80 μM decylubiquinone and 80 μM DCPIP (molar extinction coefficient=19.1 mM⁻¹cm⁻¹). Reduction in the absorbance of DCPIP was monitored at 600 nm every min for 10 min using a microplate reader.

For SDH assay, reaction was initiated by adding reaction buffer containing 400 μM phenazine methosulphate (PMS) as an exogenous electron carrier and 150 μM MTT (molar extinction coefficient=13 mM⁻¹ cm⁻¹). Change in the absorbance of MTT was monitored at 570 nm for 10 min at 1 min intervals. Citrate synthase activity assay was measured in 0.3×10⁶ cells harvested per sample and washed with PBS. Cells were resuspended in 10 mM Tris (pH 8.5) and 0.1% Triton X-100 assay buffer, containing 300 μM acetyl CoA and 100 μM 5,5′-dithiobis(2-nitrobenzoic acid) (DTNB=molar extinction coefficient 13.6 mM⁻¹ cm⁻¹). Reaction was started by the addition of 0.5 mM oxaloacetic acid and change in absorbance recorded at 412 nm wavelength every min for 10 min. SQR, SDH and CS activity measurements were performed with 100 μL sample containing 0.3×10⁶ permeabilized cells.

Complex I activity assay was performed using Abcam (ab109721) Complex I Enzyme Activity Microplate Assay Kit (Colorimetric), per instructions. Complex I specific antibodies are precoated in the microplate wells where target was immobilized. Complex I activity was determined by following the oxidation of NADH to NAD+and the simultaneous reduction of the provided dye (ε=25.9 mM⁻¹ well⁻¹) which leads to increased absorbance at OD 450 nm. Complex I activity measurements were performed with 250 μg total protein per sample resuspended in 200 μL assay buffer. Enzyme activities were calculated from the change in absorbance using the extinction coefficients of the respective dyes.

1. A method of treating cancer comprising administering an electron transport chain complex I inhibitor and/or a complex II succinate ubiquinone reductase (SQR) inhibitor in combination with another anticancer agent.

2. The method of claim 1 wherein the other anticancer is a BCL-2 antagonist.

3. The method of claim 1 wherein the cancer is a hematological malignancy.

4. The method of claim 1 wherein the electron transport chain complex I inhibitor is 5-(5-methyl-1-(3-(4-(methylsulfonyl)piperidin-1-yl)benzyl)-1H-1,2,4-triazol-3-yl)-3-(4-(trifluoromethoxy)phenyl)-1,2,4-oxadiazole (IACS-010759), its derivative, prodrug, or salts thereof.

5. The method of claim 1 wherein the BCL-2 antagonist is 2-((1H-pyrrolo[2,3-b]pyridin-5-yl)oxy)-4-(4-((4′-chloro-5,5-dimethyl-3,4,5,6-tetrahydro-[1,1′-biphenyl]-2-yl)methyl)piperazin-1-yl)-N-((3-nitro-4-(((tetrahydro-2H-pyran-4-yl)methyl)amino)phenyl)sulfonyl)benzamide (venetoclax), its derivative, prodrug, or salts thereof.

6. The method of claim 1 wherein the other anticancer is dexamethasone or melphalan.

7. The method of claim 1 wherein the other anticancer is bortezomib or carfilzomib.

8. A method of treating multiple myeloma comprising administering an effective amount of 5-(5-methyl-1-(3-(4-(methylsulfonyl)piperidin-1-yl)benzyl)-1H-1,2,4-triazol-3-yl)-3-(4-(trifluoromethoxy)phenyl)-1,2,4-oxadiazole (IAC S-010759) or salt thereof in combination with venetoclax to a subject in need thereof.

9. A method of treating multiple myeloma comprising administering an effective amount of 5-(5-methyl-1-(3-(4-(methylsulfonyl)piperidin-1-yl)benzyl)-1H-1,2,4-triazol-3-yl)-3-(4-(trifluoromethoxy)phenyl)-1,2,4-oxadiazole (IACS-010759) or salt thereof in combination with venetoclax and dexamethasone to a subject in need thereof.

10. A method of treating multiple myeloma comprising administering an effective amount of 5-(5-methyl-1-(3-(4-(methylsulfonyl)piperidin-1-yl)benzyl)-1H-1,2,4-triazol-3-yl)-3-(4-(trifluoromethoxy)phenyl)-1,2,4-oxadiazole (IACS-010759) or salt thereof in combination with melphalan to a subject in need thereof.

11. A method of treating multiple myeloma comprising administering an effective amount of 5-(5-methyl-1-(3-(4-(methylsulfonyl)piperidin-1-yl)benzyl)-1H-1,2,4-triazol-3-yl)-3-(4-(trifluoromethoxy)phenyl)-1,2,4-oxadiazole (IAC S-010759) or salt thereof in combination with bortezomib to a subject in need thereof.

12. A method of treating multiple myeloma comprising administering an effective amount of 5-(5-methyl-1-(3-(4-(methylsulfonyl)piperidin-1-yl)benzyl)-1H-1,2,4-triazol-3-yl)-3-(4-(trifluoromethoxy)phenyl)-1,2,4-oxadiazole (IACS-010759) or salt thereof in combination with carfilzomib to a subject in need thereof.
